(19) United States
(12) Patent Application Publication
(10) Pub. No.: US 2013/0303987 A1
(43) Pub. Date: Nov. 14, 2013

(54) VALVE TO PREVENT AIR ENTERING AN INTRAVENOUS CIRCUIT

(71) Applicant: Philip Stuart Esnouf, Victoria (AU)

(72) Inventor: Philip Stuart Esnouf, Victoria (AU)

(73) Assignee: CHIMDEN MEDICAL PTY. LTD., Richmond, Victoria (AU)

(21) Appl. No.: 13/883,959

(22) PCT Filed: Nov. 3, 2011
(86) PCT No.: PCT/AU2011/001407

§ 371 Date: Jul. 29, 2013

(30) Foreign Application Priority Data
Nov. 11, 2010 (AU) 2010905023

(51) Int. Cl. A61M 39/24; F16K 15/14

(52) U.S. Cl. CPC A61M 39/24; F16K 15/14
USPC 604/127; 137/859

(57) ABSTRACT

A valve for use with a container having intravenous liquid therein, the valve including: a valve body including a valve chamber formed therein; an inlet in fluid communication with the valve chamber; means for connecting the inlet, in use, to the container so that liquid can flow through the inlet to said valve chamber; an outlet having means for connection, in use, to a catheter for intravenous delivery of the liquid; the outlet including an outlet port which opens to said chamber, the port being surrounded by a valve seat; a diaphragm; mounting means for mounting the diaphragm to the body so that a peripheral edge or edge region sealingly engages the body and closes said chamber and such that the diaphragm sealingly engages the valve seat; the arrangement being such that, in use, the head of liquid within the container unseats the diaphragm from the valve seat so that the liquid flows to said outlet but when the level of liquid in the container falls to a predetermined level the diaphragm sealingly engages the valve seat to stop further flow of liquid to the outlet.

This invention relates to a valve for use with intravenous containers or circuits.

Air embolism can occur when air enters a patient's circulation via an intravenous catheter. The consequences can be significant and include stroke and death. The use of locking fittings within intravenous, giving sets and associated devices has reduced the incidence of entrainment of air when an intravenous line becomes disconnected and the patients venous pressure becomes sufficiently low (negative) to allow air to enter the circulation. This is a significant risk in the use of central venous catheters.

Up until relatively recently most intravenous fluids were contained in rigid bottles and in order for the fluid within the bottle to flow into the intravenous line by gravity a venting needle was placed in the bottle to allow air to enter the bottle as the fluid emptied. When this system was common-place nursing staff and medical personnel were always aware of the risk of air entering the giving tube once the bottle had emptied, and particular care was taken to prevent this occurrence or to deal with air in the giving set before it entered the patient's circulation. For the last 15 or 20 years most intravenous fluids are now presented in collapsible bags. For this reason there is no need to use a vent needle to allow air to enter the bags as they allow all of their contents to empty by simply collapsing. This has greatly reduced the likelihood of air entering the circuit. Many giving sets also come equipped with a pump. This facilitates the rapid infusion of fluid into the patient.

Recently a very popular intravenously administered analgesic (paracetamol) has been available. This is presented in a small glass bottle and requires the use of a venting needle in the same fashion as the older intravenous fluid bottles mentioned previously. Unfortunately the contents of these small bottles can empty quite quickly and the attendant may not notice the air entering the intravenous line. This poses the risk of air embolism. The air that has entered the line may be flushed into the patient's circulation by simply replacing the empty bottle with new fluids or by using the pump to, administer fluids.

A number of valves have been designed in the past that close when the contents of a container or burette empty these are of a float valve style and are dependent on gravity and tend to be bulky, or stick as a result of the negative pressure developed when the valve closes.

Furthermore, any valve or device that is intended to address this problem would need to be of low cost and single use. Such a device could be incorporated within the giving set or could be an addition to such a system.

According to the present invention there is provided a valve for use with a container having intravenous liquid therein, the valve including:

a valve body including a valve chamber formed therein;

an inlet in fluid communication with the valve chamber;

means for connecting the inlet, in use, to the container so that liquid can flow through the inlet to said valve chamber;

an outlet having means for connection, in use, to a catheter for intravenous delivery of the liquid;

the outlet including an outlet port which opens to said chamber, the port being surrounded by a valve seat;

a diaphragm;

mounting means for mounting the diaphragm to the body so that a peripheral edge or edge region sealingly engages the body and closes said chamber and such that the diaphragm sealingly engages the valve seat;

the arrangement being such that, in use, the head of liquid within the container unseats the diaphragm from the valve seat so that the liquid flows to said outlet but when the level of liquid in the container falls to a predetermined level the diaphragm sealingly engages the valve seat to stop further flow of liquid to the outlet.

The invention also provides an apparatus for delivery of a liquid intravenously to a subject, the apparatus including:

a container having intravenous liquid therein;

a bung, or receptacle for receiving a giving set spike, closing the container;

a valve as defined above, wherein the pointed end penetrates the bung and said inlet ports are in fluid communication with the intravenous liquid in the container, the arrangement being such that when the container is disposed with its bung downwards the head of liquid unseats the diaphragm from the valve seat so that the liquid is intravenously delivered to a subject until the level of liquid in the container falls to a level where the liquid cannot hold the diaphragm away from the valve seat and flow stops.

Preferably, said means for connecting the inlet includes an inlet spigot which is formed with a pointed end which can penetrate a bung in the container.

Preferably, the inlet spigot includes inlet passageways which extend from the valve chamber to said pointed end where they terminate in inlet ports on said pointed end.

Preferably, the diaphragm is moulded from resilient material.

Preferably, the diaphragm is moulded from silicon rubber.

Preferably, the periphery of the diaphragm is relatively thin and the centre thereof is relatively thick so that resilient flexure occurs at the periphery of the diaphragm whereas the centre moves like a solid body or disc.

Preferably, the ratio of the area of the diaphragm exposed to the chamber relative to the area of the outlet port is in the range 50 to 350.

Preferably, the ratio is about 100.

Preferably, the area of the diaphragm exposed to the chamber is about 178 sq mm and the area of the outlet port bounded by said valve seat is about 1.75 sq mm.

Preferably, the silicon rubber has a Shore A hardness in the range 10 to 30.

Preferably, the periphery of the diaphragm includes annular grooves which define an integral flange having a thickness of about 0.5 mm.

Preferably, the centre of the closure of the diaphragm has a thickness greater than about 1.7 mm.

Preferably, the body includes a cylindrical wall and a base wall which define the valve chamber and wherein the mounting means includes a mounting ring snugly received in said cylindrical wall and operable to clamp the periphery of the diaphragm against said base wall.

Preferably, the base wall includes an annular shoulder and the periphery of the diaphragm is clamped against the shoulder by the mounting ring.

Preferably, the shoulder includes a projecting rib which is received within an annular groove formed in the periphery of the diaphragm.

Preferably, the valve consists of three moulded parts: the valve body; the diaphragm; and the mounting means, the inlet, outlet and means for connecting the inlet being integrally moulded with the valve body.

Preferably, the valve body is moulded from transparent plastics material and wherein the valve is disposable.

In the valve of the invention, the diaphragm is biased so that it normally is resiliently held against the valve seat so that the valve is closed. When the valve is fitted into a full container of liquid, either in a big or bottle, or fitted in a line from a bag or bottle, it needs only a few centimeters of water pressure to unseat the diaphragm from the valve seat so that fluid can flow through the valve into the outlet where it can be delivered by catheter to the patient. When, however, the level of liquid in the container falls to a few centimeters of water, the pressure of the liquid is insufficient to hold the diaphragm away from the valve seat and the valve closes. This effectively prevents any air passing through the valve because it requires pressure of the intravenous liquid to hold the valve open.

The effect of the negative pressure below the valve that tends to hold float valve designs closed, even when there is fluid above the float, is mitigated in the valve of the invention by the large ratio of the surface area of the diaphragm acted on by the fluid head above the valve to the very small surface area of the inlet surrounded by the valve seat exposed to the negative pressure below.

Preferably further, the diaphragm includes a central projection which is accessible to a user and enables the user to grasp the projection and manually unseat the diaphragm from the valve seat.

In an alternative embodiment, an air channel is integrally formed with the outlet so as to enable air to enter the container to replace the intravenous liquid draining therefrom.

The invention will now be further described with reference to the accompanying drawings, in which:

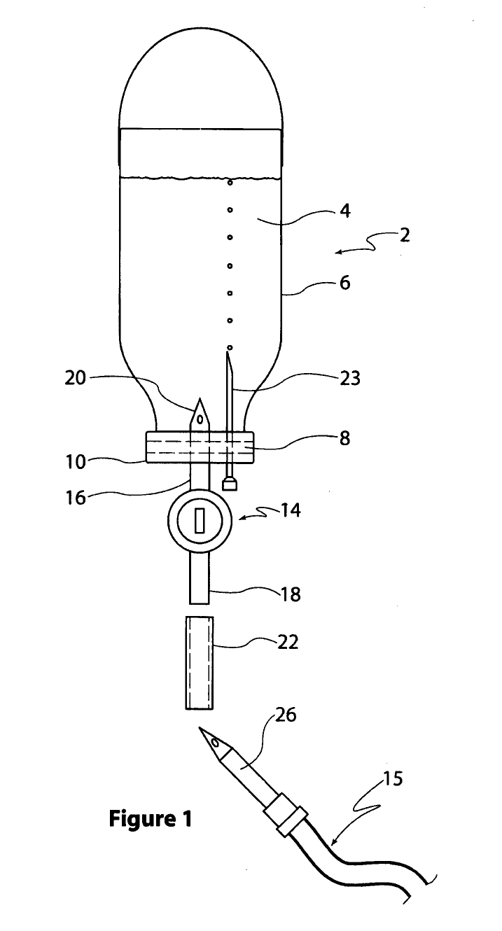

FIG. 1 is a schematic view of apparatus for intravenously delivering a liquid to a patient;

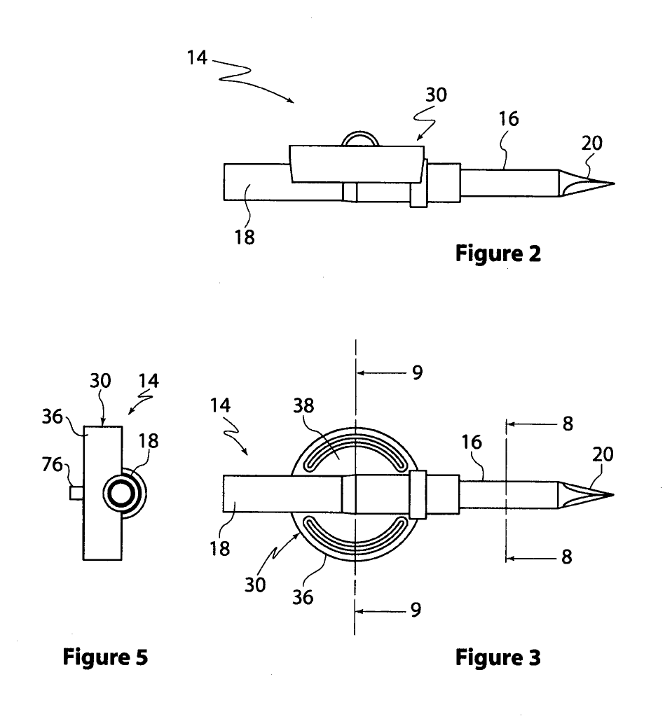

FIG. 2 is a side view of one form of valve constructed in accordance with the invention;

FIG. 3 is an underside view of the valve;

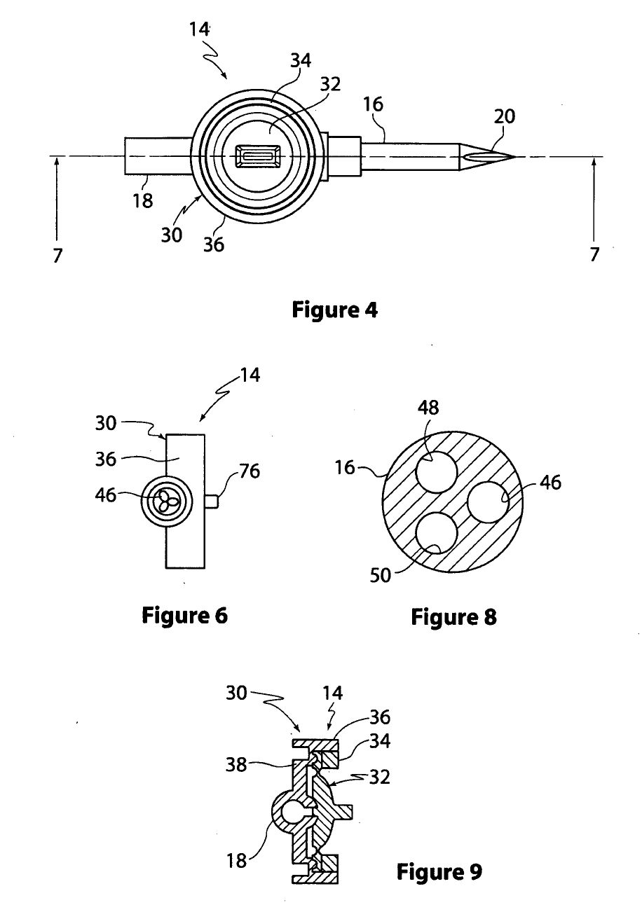

FIG. 4 is a plan view of the valve;

FIG. 5 is an end view of the valve;

FIG. 6 is another end view of the valve;

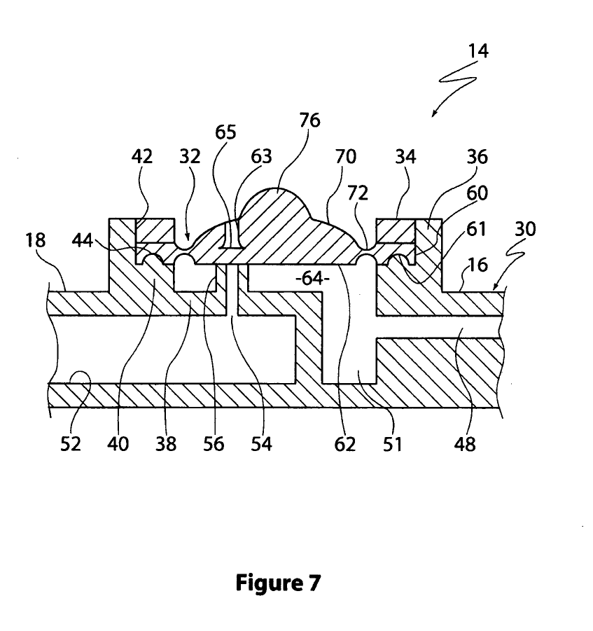

FIG. 7 is an enlarged fragmentary cross-sectional view along the line 7-7;

FIG. 8 is a cross-sectional view along the line 8-8;

FIG. 9 is a cross-sectional view along the line 9-9;

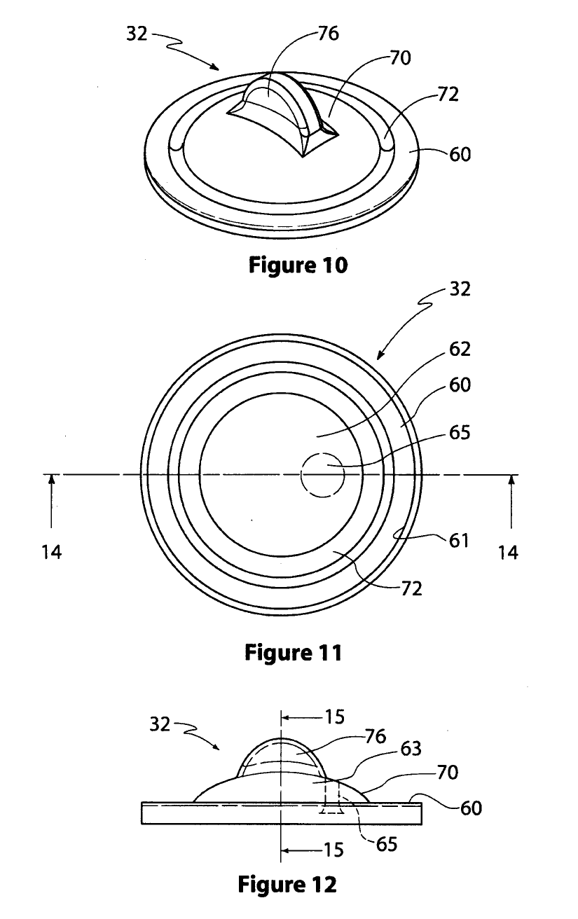

FIG. 10 is an isometric view of the diaphragm;

FIG. 11 is an underside view of the diaphragm;

FIG. 12 is a side view of the diaphragm;

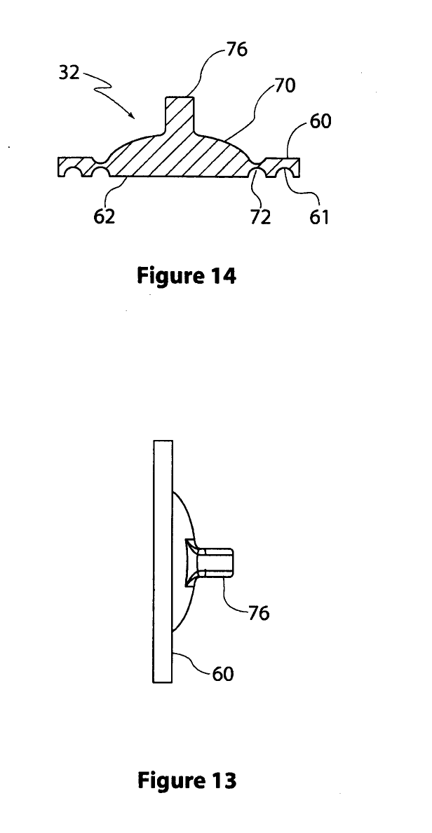

FIG. 13 is another side view of the diaphragm;

FIG. 14 is a cross-sectional view along the line 14-14;

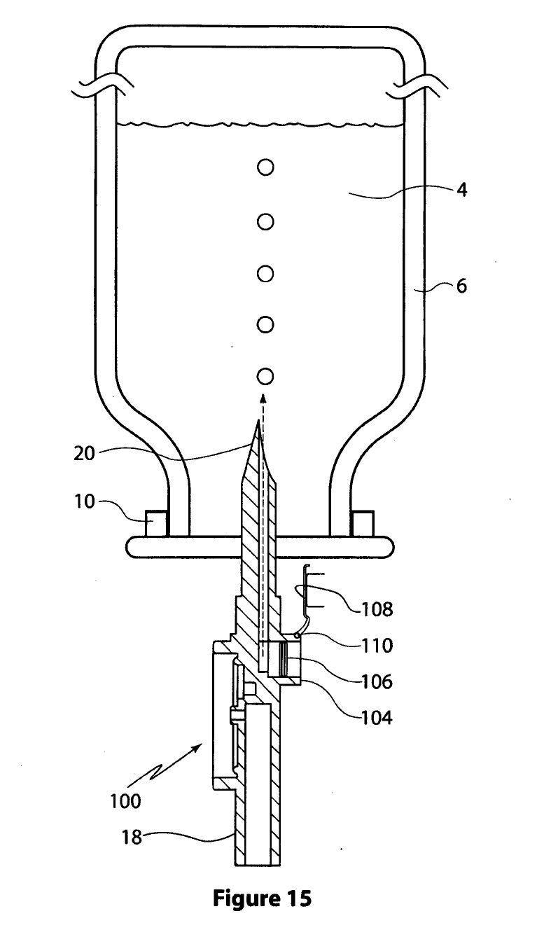

FIG. 15 is a schematic view of an apparatus for intravenously delivering liquid to a patient utilizing a modified form of valve of the invention;

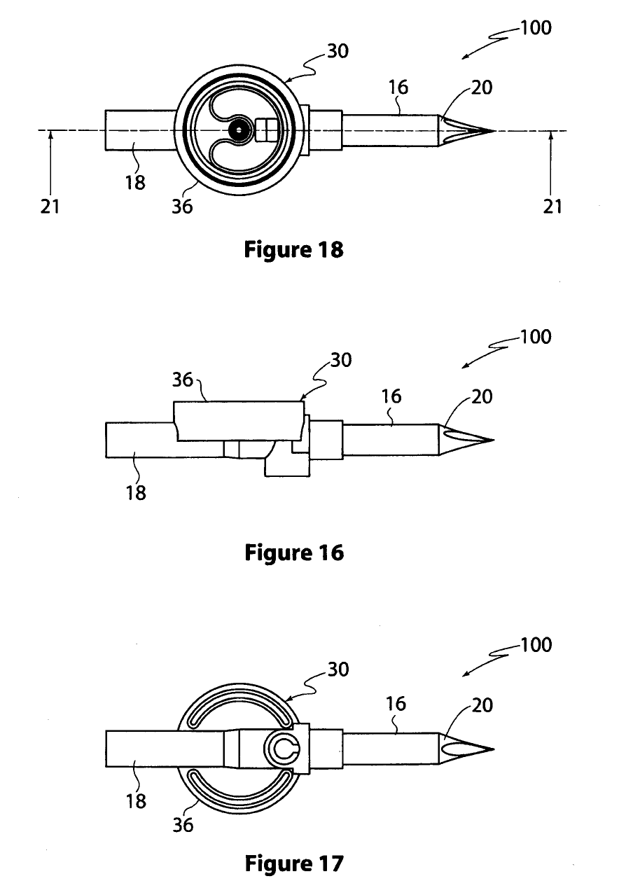

FIG. 16 is a side view of the modified form of valve;

FIG. 17 is an underside view of the valve;

FIG. 18 is a plan view of the valve;

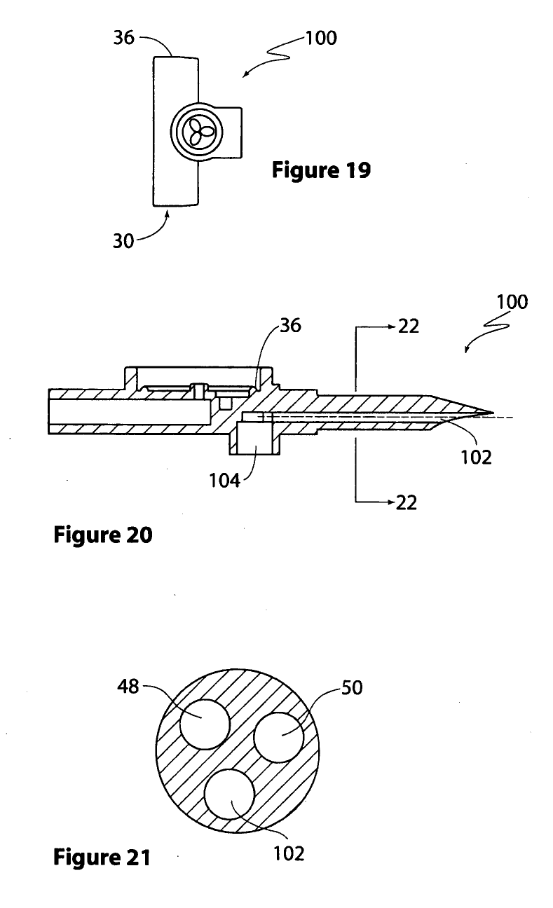

FIG. 19 is an end view of the valve;

FIG. 20 is a cross-sectional view along the line 20-20;

FIG. 21 is a cross-sectional view along the line 21-21;

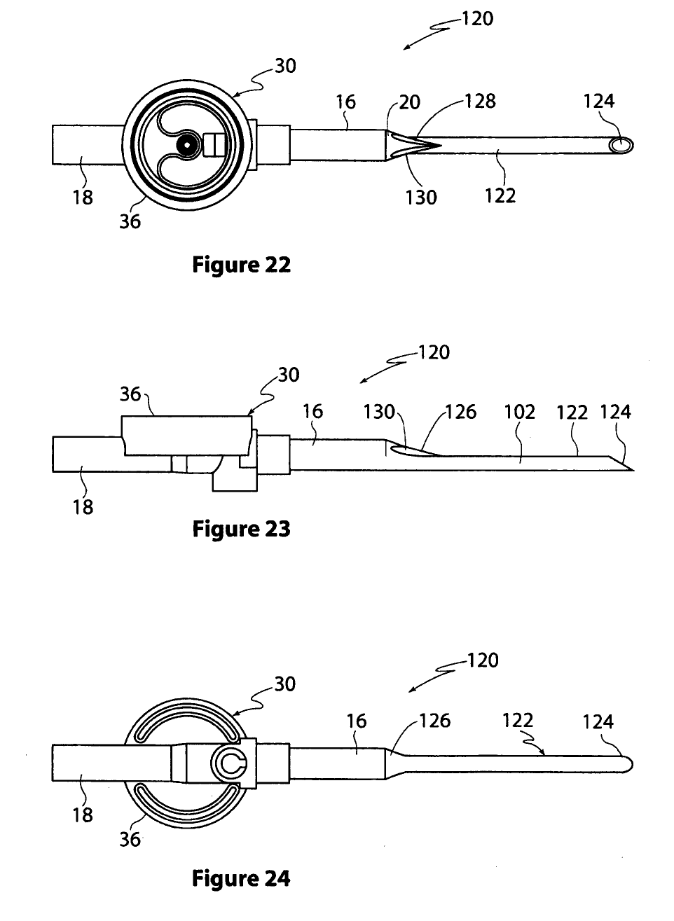

FIG. 22 is a plan view of a further modified valve of the invention;

FIG. 23 is a side view of the valve shown in FIG. 22; and

FIG. 24 is an underside view of the valve shown in FIG. 22.

FIG. 1 schematically shows apparatus 2 for delivering liquid 4 held within a container 6 intravenously to a patient (not shown). The container 6 includes a resilient bung 8 which is held in place by means of a cap 10 which includes a central opening to expose the bung. The apparatus includes a valve 14 constructed in accordance with the invention which can be coupled to a catheter 15 which has a needle (not shown) or the like for intravenous delivery of fluid to a patient in the usual way. The valve 14 includes an inlet spigot 16 and outlet spigot 18. The inlet spigot has a pointed end 20 which has sufficient rigidity that it can be pressed by a user through the resilient bung 8 so that the inlet spigot 16 is in fluid communication with the interior of the container 6. Various arrangements can be made for connecting the outlet spigot 18 to the catheter 15. In the illustrated arrangement, a polyethylene tube 22 is mounted over the spigot and has an internal diameter suitable for receiving a spike 26 which is typically provided on a giving set which forms part of the catheter 15. Typically the tube 22 has an internal diameter of say 5 mm and an external diameter of 7 mm. This provides a simple but effective way of interposing the valve 14 in an otherwise standard configuration of an intravenous giving set.

A vent needle 23 is penetrated through the bung 8 in the normal way to vent air into the container 6 as the liquid 4 flows therefrom in order to prevent build-up of negative pressure within the container.

FIGS. 2 to 9 illustrate the valve in more detail. The valve 14 preferably consists of three components, a body 30, diaphragm 32 and mounting ring 34. The body 30 is preferably injection moulded from plastics material such as transparent polycarbonate. It is integrally formed with the inlet spigot 16 and outlet spigot 18. The body 30 includes a cylindrical sidewall 36 and generally circular base wall 38. A shoulder 40 is formed between the base wall 38 and the cylindrical inner wall 42 of the sidewall 36. The shoulder 40 is formed with an annular projecting rib 44, as best seen in FIG. 7.

The pointed end 20 of the inlet spigot 16 enables the pointed end to be pierced through the resilient bung 8, as mentioned above. The inlet spigot 16 includes three longitudinally extending passages 46, 48 and 50 which open to the pointed end 20 as inlet ports and communicate with an inlet manifold 51 formed beneath the base wall 38 of the body 30. The outlet spigot 18 includes an outlet passage 52 which communicates with an outlet port 5.4 formed within a projection 56, the end face constituting an annular valve seat 58. In the illustrated arrangement, the valve seat 58 is raised approximately 0.5 mm with respect to the top surface of the shoulder 40.

The diaphragm 32 is preferably injection moulded from silicon rubber having a predetermined hardness. Typically the hardness will be in the range from 10 to 30 Shore A and preferably about 20. The actual hardness will depend on the thickness of the diaphragm, as will be apparent from the explanation below.

The diaphragm includes a peripheral mounting portion 60 which includes an annular groove 61 which is complementary to the rib 44 of the shoulder 40 so that the rib is received within the groove 61. The mounting ring 34 has a size and shape such that it is snugly received within the inner wall 42 of the sidewall 36. It serves to clamp the peripheral mounting portion 60 of the diaphragm between it and the shoulder 40. The ring has an interference fit within the inner wall 42 so that it securely retains the diaphragm 32 clamped in position.

The space between the base wall 38, lower face. 62 of the diaphragm and the annular inner wall of the shoulder 40 define a valve chamber 64. The valve chamber communicates with the ports formed at the pointed end of the inlet spigot 16 via the inlet manifold 51 and passages 46, 48 and 50. It will be seen from FIG. 7 that the normal position for the diaphragm 32 is such” that its lower, face 62 is resiliently biased, into engagement with the valve seat 58 so that the valve is normally closed.

The diaphragm 32 is formed with a cavity 63 which is somewhat wider at its lower end, as seen in FIG. 7. The cavity defines a relatively thin walled membrane 65 which is located adjacent to the valve seat 58. The relatively thin walled membrane 65 enhances the seal against the valve seat 58 because it has additional flexure and can accommodate some angular misorientation of the lower face 62 and yet still form a satisfactory seal.

The diaphragm 32 is illustrated in more detail in FIGS. 10 to 14. It will be seen that it has a central portion 70 which is substantially thicker than the peripheral mounting portion 60. The central portion 70 is joined to the peripheral mounting portion 60 by means of a resilient flange 72 defined by opposed grooves formed in the opposite sides of the diaphragm wall. The arrangement is such that when fluid pressure within the valve chamber 64 increases, resilient flexure will occur at the relatively thin flange 72 whereas the central portion 70 will be displaced by that flexure but will not itself undergo any significant resilient deformation. This maintains the lower face 62 flat so that opening and closing of the valve is better controlled compared to an arrangement where all of the diaphragm undergoes resilient flexure.

When the pressure within the valve chamber 64 increases to a predetermined level, resilient flexure will occur at the flange 72 and the central portion 70 of the diaphragm will move away from the projection 56 so that the lower face 62 of the flange will no longer sealingly engage the annular valve seat 58. When this occurs, the intravenous liquid 4 flows through the passages 46, 48 and 50 into the manifold 51 into the valve chamber 64 and then through the port 54 and finally into the outlet passage 52 of the outlet spigot 18.

As best seen in FIG. 10, the central portion 70 of the diaphragm is formed with a tab 76 which can be grasped by a user to manually unseat the diaphragm from the valve seat 58 in order to manually open the valve if that should be required.

The arrangement of the valve of the invention is such that when the pressure within the valve chamber 64 is greater than 2 or 3 cm of water, the diaphragm 32 will be unseated from the valve seat 58 and fluid flow will occur. Thus, in the arrangement of FIG. 1, when the valve 14 is located beneath a suspended container 6, the pressure of the liquid 4 within the full container easily opens the valve and flow occurs through the valve to the catheter 15 as required. When, however, the liquid 4 has nearly emptied from the container 6, the pressure will fall to a point where it will be insufficient to hold the diaphragm 32 away from the valve seat 58 and flow will cease. In the event that this occurs prematurely, an operator can grasp the tab 76 in order to manually open the valve. It will be appreciated that with the arrangement of the invention, the possibility of having air admitted to the catheter 15 is minimised.

The opening and closing of the valve is essentially dependent on the elasticity of the resilient flange 72 as well as the differential forces across the diaphragm. These differential forces depend on the relative areas of the outlet port 54 compared to the surface area of the lower face 62 of the diaphragm which is exposed to the fluid pressure within the chamber 64. Typically, the diameter of the diaphragm is in the range 20 mm to 30 mm and preferably about 25 mm but somewhat less than this is exposed to the pressure of fluid in the chamber 64 because the peripheral mounting portion 60 is not exposed. Typically, the diameter of the outlet port 54 is about 1.2 mm. Accordingly, the ratio of the surface area of the exposed part of the diaphragm relative to the cross-sectional area of the outlet port 54 is large and typically in the range 50 to 350.

As best seen in FIG. 7, the projection 56 is eccentrically mounted relative to the diaphragm 32. In use, the outlet port 54 is therefore lower down in the chamber 64 so that when the liquid first enters the chamber 64 it will tend to act on the lower part of the diaphragm 32 first and cause more reliable opening of the valve.

In the illustrated arrangement, the components are injection moulded from relatively inexpensive materials and are easily assembled. This makes the valve cheap enough to be disposable or suitable for single use.

It will be appreciated that the valve of the invention can be modified. For instance, the inlet and outlet spigots 16 and 18 could be provided with Luer connectors which are integrally moulded therewith so that the valve can be fitted in a line or catheter which extends from a supply of intravenous liquid to the patient.

FIGS. 15 to 21 illustrate a modified form of valve 100 constructed in accordance with the invention. The same reference numerals have been used to denote parts which are the same as or correspond to those of the previous embodiment.

The main difference between the valve 100 and the earlier embodiment is that it includes an air channel 102 in the outlet spigot 18 so as to perform the function of the vent needle 23. As best seen in FIG. 20, the air channel 102 extends to an air inlet port 104 which is integrally formed with the body 30. Normally the port 104 includes a filter 106, as shown in FIG. 15. A cap 108 can also be provided to close the port 104 until the device is ready to be used. In the arrangement schematically illustrated in FIG. 15, the cap 108 is moulded integrally with the body 30 and joined thereto by means of an integral hinge 110. As seen in FIG. 21, the air channel 102 is provided in the spigot 16 and effectively replaces the liquid passage 46 of the earlier embodiment.

FIGS. 22 to 24 schematically illustrate a modified valve 120. The main difference between the valve 120 and the valve 100 is that the spigot 16 is integrally formed with an elongate tube 122 having a pointed end 124. The inner bore of the tube 122 is the air channel 102. Spigot 16 includes a tapered portion 126 to facilitate penetration of the spigot 16 through the bung 8. The tapered portion 126 includes inlet ports 128 and 130 which communicate with the fluid passages 48 and 50 respectively. Preferably the tube 122 is about 70 mm to 80 mm long as measured from the tapered portion 126. The valve 120 in use has its pointed end much higher in the container 6 after it has been inserted. This therefore gives approximately 6cm to 8cm head of water pressure at the fluid ports 128 and 130 relatively to the inlet of the air channel 102. This allows the valve to open more reliably and to overcome any surface tension effects of the liquid which might prevent operation of the air channel.

Many modifications will be apparent to those skilled in the art without departing from the spirit and scope of the invention.

### LIST OF PARTS

apparatus 2
liquid 2
container 6
resilient bung 8
cap 10
valve 14
catheter 15
inlet spigot 16
outlet spigot 18
pointed end 20
tube 22
vent needle 23
spike 26
body 30
diaphragm 32
mounting ring 34
cylindrical sidewall 36
base wall 38
shoulder 40
inner wall 42
annular projecting rib 44
passages 46, 48, 50
inlet manifold 51
outlet passage 52
outlet port 54
projection 56
annular valve seat 58
peripheral mounting portion 60
annular groove 61
lower face 62
cavity 63
valve chamber 64
membrane 65
central portion 70
resilient flange 72
tab 76
valve 100
air channel 102
air inlet port 104
filter 106
cap 108
hinge 110
modified valve 120
elongate tube 122
pointed end 124
tapered portion 126
ports 128, 130

1. A valve for use with a container having intravenous liquid therein, the valve including:
a valve body including a valve chamber formed therein;
an inlet in fluid communication with the valve chamber;
means for connecting the inlet, in use, to the container so that liquid can flow through the inlet to said valve chamber;
an outlet having means for connection, in use, to a catheter for intravenous delivery of the liquid;
the outlet including an outlet port which opens to said chamber, the port being surrounded by a valve seat;
a diaphragm;
mounting means for mounting the diaphragm to the body so that a peripheral edge or edge region sealingly engages the body and closes said chamber and such that the diaphragm sealingly engages the valve seat;
the arrangement being such that, in use, the head of liquid within the container unseats the diaphragm from the valve seat so that the liquid flows to said outlet but when the level of liquid in the container falls to a predetermined level the diaphragm sealingly engages the valve seat to stop further flow of liquid to the outlet.

2. A valve as claimed in claim 1, wherein said means for connecting the inlet includes an inlet spigot which is formed with a pointed end which can penetrate a bung in the container.

3. A valve as claimed in claim 2, wherein the inlet spigot includes inlet passageways which extend from the valve chamber to said pointed end where they terminate in inlet ports on said pointed end.

4. A valve as claimed in claim 1, wherein the diaphragm is moulded from resilient material.

5. A valve as claimed in claim 4, wherein the diaphragm is moulded from silicon rubber.

6. A valve as claimed in claim 5, wherein the periphery of the diaphragm is relatively thin and the centre thereof is relatively thick so that resilient flexure occurs at the periphery of the diaphragm whereas the centre moves like a solid body or disc.

7. A valve as claimed in claim 6, wherein the ratio of the area of the diaphragm exposed to the chamber relative to the area of the outlet port is in the range 50 to 350.

8. A valve as claimed in claim 6, wherein the ratio is about 100.

9. A valve as claimed in claim 7, wherein the area of the diaphragm exposed to the chamber is about 178 sq mm and the area of the outlet port bounded by said valve seat is about 1.75 sq mm.

10. A valve as claimed in claim 5, wherein the silicon rubber has a Shore A hardness in the range 10 to 30.

11. A valve as claimed in claim 6, wherein the periphery of the diaphragm includes annular grooves which define an integral flange having a thickness of about 0.5 mm.

12. A valve as claimed in claim 11, wherein the centre of the closure of the diaphragm has a thickness greater than about 1.7 mm.

13. A valve as claimed in claim 1, wherein the body includes a cylindrical wall and a base wall which define the valve chamber and wherein the mounting means includes a mounting ring snugly received in said cylindrical wall and operable to clamp the periphery of the diaphragm against said base wall.

14. A valve as claimed in claim 13, wherein the base wall includes an annular shoulder and the periphery of the diaphragm is clamped against the shoulder by the mounting ring.

15. A valve as claimed in claim 14, wherein the shoulder includes a projecting rib which is received within an annular groove formed in the periphery of the diaphragm.

16. A valve as claimed in claim 1, characterised in that the valve consists of three moulded parts: the valve body; the diaphragm;
and the mounting means, the inlet, outlet and means for connecting the inlet being integrally moulded with the valve body.

17. A valve as claimed in claim 1, wherein the valve body is moulded from transparent plastics material and wherein the valve is disposable.

18. A valve as claimed in claim 1, wherein the diaphragm is circular and wherein the valve seat engages the diaphragm at an eccentric location which, in use, is lower than the centre of the diaphragm.

19. A valve as claimed in claim 1, wherein the diaphragm includes a relatively thin walled portion which sealingly engages the valve seat.

20. A valve as claimed in claim 1, including an air channel.

21. A valve as claimed in claim 20 wherein the air channel is located within a tube having a pointed end, the tube being integrally formed with the valve body.

22. Apparatus for delivery of a liquid intravenously to a subject, the apparatus including:
a container having intravenous liquid therein;
a bung, or receptacle for receiving a giving set spike, closing the container;
a valve as claimed in claim 3 as appended directly or indirectly to claim 3, wherein the pointed end penetrates the bung and said inlet ports are in fluid communication with the intravenous liquid in the container, the arrangement being such that when the container is disposed with its bung downwards the head of liquid unseats the diaphragm from the valve seat so that the liquid is intravenously delivered to a subject until the level of liquid in the container falls to a level where the liquid cannot hold the diaphragm away from the valve seat and flow stops.
